(19) United States
(12) Patent Application Publication
(10) Pub. No.: US 2020/0268005 A1
(43) Pub. Date: Aug. 27, 2020

(54) PELLETED RUMINANT FEED ENRICHED WITH RUMEN-AVAILABLE INGREDIENTS

(71) Applicant: BASF SE, Ludwigshafen Am Rhein (DE)

(72) Inventors: Arnulf TRÖSCHER, Weinheim (DE); Uwe OBERFRANK, Romerberg (DE)

(21) Appl. No.: 16/804,086

(22) Filed: Feb. 28, 2020

Related U.S. Application Data
(62) Division of application No. 15/124,403, filed on Sep. 8, 2016, now abandoned.

(30) Foreign Application Priority Data
Mar. 13, 2014 (EP) 14159496.0

(51) Int. Cl. A23C 9/14; A23K 40/25; A61K 31/201; A23K 40/10; A23K 40/20; A23K 20/158; A23K 50/10; A61K 31/202; A61K 45/06; A61K 31/20; A61K 9/00

(52) U.S. Cl. CPC A23C 9/14; A23K 40/25; A61K 31/201; A23K 40/10; A23K 40/20; A23C 2230/10; A23K 50/10; A61K 31/202; A61K 45/06; A61K 31/20; A61K 9/0053; A23K 20/158

(57) ABSTRACT

The invention relates to pelleted ruminant feed, comprising, in pelleted or else subsequently re-crumbled form, a mixture of at least one solid particulate feed component with at least one rumen-unstable constituent which has been added to the mixture; to processes for their preparation and to their use in methods of altering the milk fat concentration.

### RELATED APPLICATIONS

This application is a divisional application of U.S. application Ser. No. 15/124,403 filed Sep. 8, 2016, which is incorporated by reference. U.S. application Ser. No. 15/124,403 is a national stage application (under 35 U.S.C. § 371) of PCT/EP2015/055006, filed Mar. 11, 2015, which claims benefit of European Application No. 14159496.0, filed Mar. 13, 2014, which is incorporated herein by reference in its entirety.

The invention relates to pelleted ruminant feed, comprising, in pelleted form, a mixture of at least one solid particulate feed component with at least one rumen-labile constituent which has been added to the mixture; to processes for their preparation and to their use in methods of altering the milk fat concentration.

### BACKGROUND OF THE INVENTION

Ruminant feed additives may be subject to undesired chemical modifications by rumen organisms. In the context of the modification of polyunsaturated fatty acids by the rumen microorganisms, for example, it can be observed that both the content and the position of double bonds in the carbon chain of the fatty acid may be altered, Usually, the double bonds of the unsaturated fatty acids in the plants are separated by at least two single bonds. Microbial enzymes are capable of generating a conjugated double-bond system in which the double bonds are separated by only one single bond. These include, for example, the conjugated linoleic acid isomers (conjugated linoleic acids, CLA). They are formed mainly by the linoleic acid isomerase enzyme of the rumen bacterium Butyrivibrio fibrisolvens, starting from linoleic acid (C18:2 cis-9, trans-12). The main CLA isomer is cis-9, trans-11-CLA (C18:2 cis-9, trans-11), which amounts to approximately 75-92% of the total CLA content. In contrast, the milk-fat-depressing isomer C18:2 trans-10, cis-12 is present in a small amount of 0.03-1.5% only. Some of these isomers reach the ruminant's organism via absorption. In the case of dairy cows, the rumen-modified fatty acids are also transported into the milk. The CLA content of the milk fat is greatly feed-dependent and amounts to approximately 0.3-1.1% (cf. Kirchgeßner, Tierernährung, 13th edition, DLG Verlag GmbH), in pasture-fed animals even up to over 4% (Troscher et al., 2014; in print).

Owing to their potent physiological or pharmacological activities, CLAs are intensively researched. CLAs show cancer-protective and antiinflammatory activities in cell culture studies and animal experiments. It is also known that conjugated linoleic acid isomers (CLA isomers) inhibit, in ruminants, the expression of a large number of genes which are responsible for the uptake, into the mammary gland, of circulating fatty acids which are responsible for the synthesis of fatty acids and the formation of triglycerides. It is in particular the C18:2 trans-10, cis-12 CLA isomer which has proved to be particularly fat-reducing in this context. Advantages which are discussed in connection with the reduction of the milk fat content are, inter alia, relief of the metabolism and increased milk yields. It has been reported that a reduction of the milk fat content of 0.4-0.5% may result in increased meal yields by up to 3-10% (cf. Kirchgeßner, loc. cit.).

This is why CLAs are currently often added to milk production ration. While these preparations are approved in feeding, they have to be employed in rumen-protected form since unprotected CLAs are further degraded in the rumen into ineffective C18:1 and C18:0 fatty acids.

When these or other molecules which are degraded or chemically modified by rumen micro-organisms so that they lose their biochemical activity are to be administered to ruminants, they will, therefore, have to be protected against such undesired microbial activities in the rumen. The literature describes various methods which are capable of imparting rumen protection to polyunsaturated fatty acids (PUFAs), such as the CLAs. This is also clear from findings, for example by Elgersma et al., in Fresh Herbage for Dairy Cattle, 175-194, 2006, which describe biohydrogenation rates of unsaturated fatty acids in the range of 82-98%. Only approximately 2 to 22% of the polyunsaturated fatty acids (PUFAs) which have been ingested can still be detected after passing through the rumen.

Thus, the literature (cf. Kirchgeßner, loc. cit.) describes various forms of rumen-protected fats which are protected against microbial degradation and microbial modification:

Mentioned are in particular:

a) native protected cell-bound oilseed fats, which are employed in the form of oilseed cake or crushed whole seeds. The principle of the protection here is the slow release of the oils from the plant cells; however, the storage stability of such fats is poor.
b) heat-treated oilseeds, for example by extrusion; here, denatured proteins surround the fat droplet, thus protecting it from microbial degradation.
c) chemically modified fats, for example by suponification of fatty acids with calcium or coating the fat droplets with formaldehyde-treated proteins.
d) Fats which are protected from microbial enzymes by technical methods (for example coating with hardened vegetable fats).

EP-A-1 100 489 discloses a method for reducing the milk fat content in dairy cattle, where an effective amount of CLA is to be administered to the cattle. By way of protection against modification from rumen bacteria, it is proposed to administer the active substance by injection or to provide it in coated form.

### DESCRIPTION OF THE FIGURE

FIG. 1 shows the retention profile over time of various in-vitro incubated CLA formulations in pellets and extrudates (T1 and T3 according to the invention).

### SUMMARY OF THE INVENTION

The object of the invention is to provide a simplified route for converting rumen-unstable feed components into a dosage form with improved rumen stability.

Surprisingly, the object was achieved by providing uncoated, pelleted feeds as per the appended claims.

### SPECIFIC EMBODIMENTS OF THE INVENTION

### a) General Definitions

A “pellet” or a “pelleted form” comprises solid feed formulations as are obtainable in a manner known per se with the aid of conventional pelleting devices, but also conventional extrusion or expanding devices. Usually, such pellets have a length of approximately 0.5 to 2 or 0.8 to 1.5 cm, such as, for example, approximately 1 cm, and a diameter in the range of from 0.1 to 0.8 or 0.4 to 0.5 cm. Also encompassed by these terms are what are known as “crumbled” pellets, i.e. pellets which are converted into smaller particles by the action of mechanical force. The purpose of crumbling is frequently to make it easier for the animal to take up the pellets, or else better distribution in the final feed. Crumbling allows the particle size to be reduced in a targeted manner and the number of particles to be increased, giving, for example, a particle mixture with a content of >50%, for example 55 to 95 or 60 to 90 or 70 to 80% of particles with a diameter in the range of from approximately 3 to 6 or 4 to 5 mm.

A “rumen-unstable constituent” (pure substances, but also mixtures of natural or synthetic substances) comprises a chemical compound which, without sufficient protection, may be subjected to chemical modifications (modification of the chemical empirical formula, such as, for example, by biohydrogenation, and/or changes in configuration and/or stereochemistry) upon passing through the ruminant's rumen. As a rule, this will be an organochemical substance, in particular one which is of importance as a food/feed component or a food/feed additive. Those which must be mentioned in particular are saturated or mono- or polyunsaturated carboxylic acids, such as, for example, those with at least 6 carbon atoms, such as e.g. the CS, PUFA, MUFA or CLA stated below. Further feed additives, or feed components or straight feeds which must/may be administered in rumen-protected form are amino acids (in particular methionin, lysine and the like), enzymes, cholin and other active substances from the class of the vitamins.

A “rumen-unstable mixture” or “rumen-unstable feed mixture” comprises at least one “rumen-unstable” constituent and is not or insufficiently rumen-protected, i.e. the constituent, if fed and passing through the ruminant's rumen, would be exposed to chemical changes as described above.

In the context of the present invention, “rumen stability” or “rumen protection” means that a “rumen-unstable” constituent is converted by the above-described “pellet” form into a state of reduced “rumen instability” up to an essentially complete rumen protection. This improved rumen stability or reduced rumen instability can be determined in a simple manner by comparing the stability of the constituent in a pelleted or nonpelleted mixture using experimental approaches (in vitro or in vivo) which are described in the experimental part.

“Carboxylic acids” (CS) are, in particular, straight-chain or branched, in particular straight-chain, saturated or mono- or polyunsaturated, optionally substituted C₆-C₃₀-monocarboxylic acids. Examples of saturated unbranched fatty acids are caproic acid, oenanthic acid, caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachidic acid, behenic acid, lignoceric acid, cerotic acid and melissic acid. Examples of monounsaturated fatty acids are palmitoleic acid, oleic acid and erucic acid. Examples of diunsaturated fatty acids are sorbic acid and linoleic acid. Examples of triunsaturated fatty acids are linolenic acid and elaeosteric acid. Examples of tetra- and polyunsaturated fatty acids are arachidonic acid, clupanodonic acid, eicosapentaenoic acid and docosahexaenoic acid. Examples of substituted fatty acids are ricinoleic acid ((R)-12-hydroxy-(Z)-9-octadecenoic acid). Further suitable fatty acids are naturally occurring fatty acids such as gondo acid and neronic acid. If double bonds are present in the fatty acids, they may be present both in cis and in trans form. The substituents are preferably selected among hydroxyl and lower alkyl groups, such as, for example, methyl and ethyl groups. Keto groups or epoxy groups may furthermore be present in the hydrocarbon radical, as is the case for example in vernolic acid. Further functional groups are cyclopropane, cyclopropene and cyclopentene rings, which may be formed by bridging of two adjacent carbon atoms in the hydrocarbon radical of the fatty acid (cf. malvalic acid and chaulmoogric acid).

“PUFAs” are polyunsaturated fatty acids with at least two conjugated or nonconjugated C═C double bonds in the fatty acid molecule. Examples which may be mentioned are: linolenic acid, eicosapentaenoic acid (EPA) ((5Z,8Z,11Z,14Z,17Z)-eicosa-5,8,11,14,17-pentaenoic acid; or C20:5 (ω-3)) and docosahexaenoic acid (DHA) ((4Z,7Z,10Z,13Z,16Z,19Z)-docosa-4,7,10,13,16,19-hexaenoic acid) or C22:6 (ω-3)).

“MUFAs” are monounsaturated fatty acids which may be present in cis or trans configuration, such as, for example, oleic acid or vaccenic acid.

Conjugated linoleic acids (CLA) are a group of isomers of the C₁₈-monocarboxylic acid “linoleic acid”, which is diunsaturated and whose two double bonds are in positions 9 and 12 and are, therefore, not conjugated. Linoleic acid is also designated by the abbreviation “018:2 cis-9, cis-12”.

“CLA” comprises, in principle, all conjugated, diunsaturated isomers of linoleic acid (C18:2 cis-9,cis-12), where the position of the two double bonds in the carbon chain may be shifted towards the chain end or towards the carboxyl group and where the stereochemistry of the conjugated double bonds may furthermore comprise any variation (cis/cis, trans/trans, cis/trans), where “cis/trans” comprises the two sequences “trans-cis” or “cis-trans”, with the first-mentioned configuration of the two sequences in each case relating to the double bond which is closest to the carboxyl group:

By way of example, reference may be made to the conjugated linoleic acid “C18:2 cis-9, trans-11”, which is shown hereinbelow:

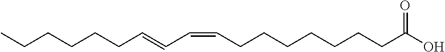 [SMILES: CCCCCC/C=C/C=C\CCCCCCCC(=O)O]

Therefore, the names cis/trans-9,11-linoleic acid, cis/trans-8,10-linoleic acid, cis/trans-11,13-linoleic acid, and cis/trans-10,12-linoleic acid, used herein comprise both the cis-trans and the trans-cis isomers, in other words:

cis/trans-9,11-linoleic acid comprises: C18:2 cis-9, trans-11 and C18:2 trans-9,cis 11
cis/trans-8,10-linoleic acid comprises: C18:2 cis-8, trans-10 and C18:2 trans-8,cis 10
cis/trans-11,13-linoleic acid comprises: C18:2 cis-11, trans-13 and C18:2 trans-11,cis 13
cis/trans-10,12-linoleic acid comprises: C18:2 cis-10, trans-12 and C18:2 trans-10,cis 12

The same applies analogously to linolenic acid, which is triunsaturated (C18:3, cis-9, cis-12, cis-15) and its cis/trans isomers.

The above information on the substances and the uses described herein of these substances primarily relate to the respective pure substance, but also to natural or synthetic substance mixtures which comprise at least one of these substances, for example at least one PUFA or at least one CLA.

Natural substance mixtures are, for example, fish oils or microbial oils, which may be high in PUFAs, and linseed oil, soya oil, sunflower oil, castor oil and the like.

Synthetic substance mixtures are, for example, commercial products which are high in CLA, such as Lutalin® by BASF SE.

Others which must be mentioned are rumen-unstable derivatives of the abovementioned carboxylic acids (CS, PUFA, MUFA, CLA), such as, in particular, substituted derivatives. Examples which must be mentioned are compounds which are mono- or polysubstituted on the hydrocarbon radical of the carboxylic acid, such as, for example, by hydroxyl groups. An example of such a compound which must be mentioned is: 10-hydroxy-cis-12-octadecadienoic acid.

### b) Specific Embodiments

The present invention relates in particular to the following:
1. A pelleted ruminant feed, comprising, in pelleted form (i.e. fairly small particles obtained by pelleting, extrusion, expansion but also in crumbled form, i.e. by the subsequent crumbling of pellets, extrudates or expandates), a mixture of at least one solid particulate feed component with at least one rumen-labile constituent which has been added to the mixture (in pure form or as a component of a natural or synthetic substance mixture), (rumen-unstable) where in particular individual, several or all mixture components which are to be mixed for pelleting with the rumen-unstable constituent will, per se, not demonstrate any, or no sufficient, rumen-stabilizing activity in the mixture to be pelleted. In particular, for example in CLA-containing mixtures to be pelleted, such mixture components are present not at all or only in an ineffective amount, which bring about a rumen stabilization of the CLA in unpelleted CLA mixtures. Examples which must be mentioned in this context are the additives with a stabilizing activity which are present in the rumen-stabilized commercial CLA product Lutrell®, such as, in particular, soya oil, gypsum or silica, which are present in feed mixture according to the invention which is to be pelleted not at all or only in non-stabilizing amounts.
   The mixture employed for preparing the feed pellets can therefore be referred to as a “rumen-unstable” mixture, which is only conferred “rumen stability” i.e. whose rumen instability is reduced, by the pelleting, extrusion or expansion step according to the invention.
   As a consequence, it is preferred in accordance with the invention that no feed mixtures be processed to pellets which already have sufficient rumen stability prior to pelleting. For example, the invention therefore does not comprise the processing of rumen-protected constituent mixtures (such as, for example, the CLA mixture Lutrell® or mixtures of comparable compositions) to give pelleted ruminant feed.
2. A feed according to embodiment 1, wherein the constituent is a fat or oil, in particular a fatty acid (carboxylic acid with at least 6 carbon atoms).
3. A feed according to any of the preceding embodiments, where the amount of added rumen-labile constituent is in the range of from 0.1 to 20, 1 to 15, 2 to 10 or 3 to 5% by weight, based on the total weight of the mixture to be pelleted.
4. A feed according to any of the preceding embodiments, wherein the constituent is selected from among saturated and mono- or polyunsaturated (especially diunsaturated) carboxylic acids with at least 6 carbon atoms, mixtures of at least two saturated and/or mono- or polyunsaturated (especially diunsaturated) carboxylic acids with at least 6 carbon atoms, and substance mixtures which comprise at least one saturated or mono- or polyunsaturated (especially diunsaturated) carboxylic acid with at least 6 carbon atoms, such as e.g. a C18:2 or C18:3 carboxylic acid; substituted derivatives of such carboxylic acids, such as e.g. hydroxyl-substituted derivatives, for example 10-hydroxy-cis-12-octadecadienoic acid, are also included.
5. A feed according to any of the preceding embodiments, wherein the constituent comprises at least one polyunsaturated (especially polyunsaturated) fatty acid (PUFA), in particular with conjugated double bonds.
6. A feed according to embodiment 5, wherein the polyunsaturated (especially diunsaturated) fatty acid comprises at least one isomer of linoleic acid.
7. A feed according to embodiment 6, wherein the polyunsaturated fatty acid is a CLA.
8. A feed according to embodiment 7, wherein the CLA is selected from among
   a) cis/trans-9,11-linoleic acid,
      i.e. C18:2 cis-9, trans-11 and C18:2 trans-9,cis 11
   b) cis/trans-8,10-linoleic acid;
      i.e. C18:2 cis-8, trans-10 and C18:2 trans-8,cis 10
   c) cis/trans-11,13-linoleic acid;
      i.e. C18:2 cis-11, trans-13 and C18:2 trans-11,cis 13
   d) cis/trans-10,12-linoleic acid;
      i.e. C18:2 cis-10, trans-12 and C18:2 trans-10,cis 12
   e) cis/cis-9,11-linoleic acid,
   f) trans/trans-9,11-linoleic acid,
   g) cis/cis-8,10-linoleic acid
   h) trans/trans-8,10-linoleic acid
   i) cis/cis-11,13-linoleic acid
   j) trans/trans-11,13-linoleic acid
   k) cis/cis-10,12-linoleic acid
   l) trans/trans-10,12-linoleic acid; and mixtures of at least two of the abovementioned compounds.
9. A feed according to embodiment 8, wherein the CLA is selected from among
   a) 9-cis,11-trans-linoleic acid (C18:2 cis-9, trans-11);
   b) 10-trans,12-cis-linoleic acid (C18:2 trans-10,cis 12); and
   c) mixtures of these.
10. A process for the preparation of a pelleted ruminant feed according to any of the preceding embodiments, wherein
   a. at least one solid particulate feed component is mixed with at least one rumen-labile constituent according to the definition above,
   b. the resulting mixture is optionally conditioned, in particular by the introduction of steam, in particular conditioning with hot (e.g. up to 95° C.) steam, such as e.g. saturated steam, over a sufficient period of time, such as e.g. for 20 to 40 or 20 to 120 or 20 to 140 or 20 to 240 seconds,
   c. the optionally conditioned mixture is compressed in an extruding, expanding or a pelleting device, especially a pelleting or extruding device, with the action of pressure to give pellets; and
   d. the resulting pellets are optionally cooled and/or subjected to a final drying process.
11. A method of altering the milk fat concentration in milk which is produced by a lactating ruminant, where the lactating ruminant is provided with an effective amount of a pelleted ruminant feed according to any of embodiments 1 to 9 or prepared according to embodiment 10.
12. A method according to embodiment 11, wherein the lactating ruminant is selected from among cow, goat and sheep.
13. A method for preparing a rumen-protected ruminant feed of a rumen-unstable constituent, wherein
   a. at least one solid particulate feed component is mixed with at least one rumen-unstable constituent, which gives a rumen-unstable mixture (i.e. a mixture which would display no, or only insufficient, rumen protection of the unstable constituent) of the constituent with the at least one feed component,
   b. the resulting mixture is optionally conditioned,
   c. the optionally conditioned mixture is compressed in a pelleting, extruding or expanding device with the action of pressure and optionally steam to give pellets, where the pelleted mixture displays an improved rumen stability of the constituent in comparison with the non-pelleted mixture (i.e. displays a reduced rumen instability of the constituent up to a preferably essentially fully rumen protection); and
   d. the pellets thus obtained are optionally cooled and/or subjected to a final drying process and optionally subsequently crumbled.

14. Feed according to one of the embodiments 1 to 9, prepared by a method according to embodiment 13.

### d) Further Embodiments

### d1) Preparation of the Feed Pellets

To prepare the pelleted feed compositions, the rumen-unstable constituent(s) to be formulated in accordance with the invention are mixed, optionally together with further added customary animal feed components (such as, for example, milk production ration). The amount of constituents is chosen such that it is for example in the range of from 0.1 to 20, 1 to 15, 2 to 10 or 3 to 5% by weight. Thereafter, the feed is pelleted with the aid of a suitable pelleting press. To this end, the feed mixture is usually conditioned by passing in steam (with hot steam of a temperature of up to 95° C., for example saturated steam), for example for 20 to 1240 seconds. The increase in temperature during the pelleting process may be controlled via the added amount of saturated steam. This steam conditioning may however also be omitted.

Pelleting is usually carried out in commercially available pelleting presses (for example from Bühler GmbH), which may be designed as an annular die press. Here, rollers press the pre-conditioned substance mixture through an annular die (gap width between roller and die for example 1 mm). Depending on the die, pellets of approximately 2 to 12 mm in diameter may thus be prepared. For dairy cattle feed, one will use, for example, dies with a bore diameter of 4 to 5 mm and a slot length of 40 to 50 mm. The highest process temperature prevails when the mixture is pressed through the die. Temperatures in the range of approximately 60 to 100° C. may be reached at this stage.

The hot material which leaves the die outlet is cut off continuously by means of blades, and the pellets thus formed are immediately cooled in a cooler and optionally subjected to a final drying process. The residual moisture content may, after cooling and optionally drying, be in the range of approximately 1 to 15% by weight, based on the total pellet weight.

Alternatively, the pelleting may be replaced by extrusion or expansion (for example by means of feed expanders from Kahl, Germany) (cf., for example, “Feed Manufacturing Technology IV” Ed: McEllhiney, Kansas State Univ.; Pub. by American Feed Industry Assoc, 1994; Arlington, Va. in which may be found: Extrusion Cooking Systems, Bob Hauck et al., p. 131-139; Wilson et al. 1998, Journal of Poultry Science, 77 (Supp1.1): 41). The products of these processes may then be crumbled.

### d2) Application

Typical feed components which are useful in the production of pellets according to the invention comprise straight feeds of vegetable or animal origin as per Futtermittelverordnung (FMV; German Feed Regulation), such as, for example, cereal by-products, wheat bran shorts, wheat bran; extracted meals, pomace, dried molassed beet pulp, fish meal, meat-and-bone meals; and/or straight mineral feeds as per FMV, such as, for example, carbonates, phosphates, sulfates, propionates. Others which are suitable are cereals such as wheat, rye, barley, oats, corn, millet or triticale; cereal by-products (by-products of milling) such as brans, feeds, wheat feeds, bran shorts or middlings; by-products of oil production (extracted meals, expellers, cake); by-products of sugar production (molasses, dry beet chips, feeding sugar, pulp, potato starch, maize gluten, wheat gluten); by-products of the fermentation industry, brewer's grains, yeast, malt culms, stillage; and feedstuffs of animal and other origin, such as blood meal, fish meal, pressed must, potato protein.

Those which must be mentioned in particular are wheat, grain corn, barley, oats; soybeans; cereal meals such as wheat or corn meal, soybean meal, molassed beet chips, wheat feed, wheat middlings, corn gluten feed, extracted soybean meal, extracted rapeseed meal, brewer's grains, cereal stillage, beet molasses, oat bran; sugars such as glucose and sugar alcohols, furthermore proteinaceous components such as soybean concentrate, fish meal, glutens such as corn or wheat gluten, oils and fats, and nutraceuticals such as, for example, free amino acids, their salts, vitamins (such as, for example, A, D, E) and trace elements (such as, for example, Cu as CuSO₄), mineral constituents such as calcium carbonate, sodium chloride; phosphates and optionally processing aids, for example glidants, inert fillers and the like, and optionally preservatives.

Typical milk production ration compositions comprise, for example, corn, wheat, barley, oats, rye, citrus pomace, extracted soybean meal, extracted rapeseed meal, soybean husks, palm kernel expellers, DDGS, corn gluten feed, sugarbeet chips, wheat feeds, wheat bran, linseed, molasses, lime, salt, vitamin and trace element premix.

The invention will now be illustrated in greater detail with reference to the following use examples.

### EXPERIMENTAL PART

### Example 1: Study of the Milk Fat Depression when Feeding Dairy Cattle with Various Pelleted CLA Feed Formulations

### a) Pelleting

Pelleting press: Manufacturer Simon Heessen, Type V3-30C.
Capacity 600-650 kg/h

### Die:

Bore diameter: 5 mm
Bore length 45 mm.
Gap width 1 mm
   Pelleting temperature: 80° C.
   Conditioning: Steam T=135 and 145° C. (depending on the pressure)

### b) CLA Formulations Employed

(1) Lutalin®: (CLA-containing commercial product)

TABLE
Composition of 5 different batches

| Fatty acid | | 1 | 2 | 3 | 4 | 5 |
|---|---|---|---|---|---|---|
| Palmitic acid | % ¹ | 4 | 4 | 5.3 | 6.4 | 6.5 |
| Stearic acid | % | 3.6 | 3.6 | 3.6 | 3.5 | 3.6 |
| Oleic acid | % | 28.4 | 28.4 | 26.5 | 23 | 22 |
| Linoleic acid | % | 1.5 | 1.6 | 2.3 | 1.7 | 1.8 |
| CLA trans-10, cis-12, omega-6 | % | 29.9 | 29.9 | 29.6 | 30.4 | 31.0 |
| CLA cis-9,trans-ll | % | 29.9 | 29.9 | 29.6 | 30.4 | 31.0 |
| CLA total | % | 59.8 | 59.8 | 59.2 | 60.8 | 62.0 |
| CLA trans, trans | % | 0.4 | 0.4 | 0.4 | 0.6 | 0.4 |
| Peroxide number, Ph. Eur. (B 057) | ppm | 17.9 | 6.3 | 7.1 | 13.5 | 11.9 |
| Acid number (ISO 660-96) | mg KOH/g | 14.0 | 14.1 | 14.0 | 14.9 | 12.9 |
| Water (ISO 4317) | % | 0.050 | 0.077 | 0.049 | 0.013 | 0.184 |
| Methanol (NF EN 14110) | ppm | 40.0 | 70.0 | 40.0 | 10.0 | 40.0 |
| Total fatty acids | | 97.7 | 97.8 | 97.3 | 96.0 | 96.3 |

¹ GC area %

### (2) Lutrell

Lutrell is composed of 34% Lutalin and the additives 15% silica, 5% gypsum and 46% hydrogenated soya oil, with the rumen-stabilizing function being effected mainly by the latter.

The composition (fatty acid profile) of Lutrell is shown in the following table

| | Amount |
|---|---|
| Fatty acid | (% of the entire product) |
| C14:0 | 0.12% |
| C15:0 | 0.00% |
| C16:0 | 7.94% |
| C16:1 | 0.04% |
| C17:0 | 0.00% |
| C18:0 | 41.05% |
| C18:1, c9 | 8.18% |
| C18:2, c9, c12 | 0.67% |
| C20:0 | 0.38% |
| C18:3 | 0.00% |
| CLA cis-9, trans-11 | 9.39% |
| CLA trans-10, cis-12, | 9.45% |
| C22:0 | 0.36% |
| C24:0 | 0.12% |
| Other fatty acids | 2.42% |
| Other substances | 20.00% (inorganic carrier) |
| Σ | 100% |

(3) Lutalin plus Silafett

Fatty acid composition of Silafett=hydrogenated soya oil

| | |
|---|---|
| C12:0 | 0-0.5% |
| C14:0 | 0-1% |
| C16:0 | 7-14% |
| C18:0 | 85-93% |
| C18:1 | 0-3% |
| C18:2 | 0-0.5% |
| Trans fatty acids: | max. 1% |

### b) Experimental Design

Four experimental variants (see table hereinbelow) are tested.

Variant 1 (T1) is the control (C) and does not comprise any CLA,
Variant 2 (T2) comprises the CLA formulation “Lutrell”,
Variant 3 (T3) comprises the CLA formulation “Lutalin” and
Variant 4 (T4) comprises the CLA formulation “Lutalin plus Silafett”.

| | T1 | T2 | T3 | T4 |
|---|---|---|---|---|
| Soya/corn mix | 1000¹⁾ | 950 | 983.3 | 950.0 |
| Lutrell | 0 | 50 | 0.0 | 0.0 |
| Lutalin | 0 | | 16.7 | 16.7 |
| Lutalin plus Silafett | 0 | | 0.0 | 33.3 |
| | 1000 | 1000 | 1000 | 1000 |

¹⁾Data in grams
Feed: 1 kg of supplement composed of 25% extracted soya meal and 75% corn meal, prepelleted

The CLA preparations are mixed into a milk production ration (25% by weight extracted soybean meal; 75% by weight corn meal) and the mixture is pelleted.

The experimental design corresponds to a 4×4 Latin square (see the following table). In the test groups, the pure CLA is metered in identical amounts of in each case 16.7 g per cow per day.

| | Group 1 | Group 2 | Group 3 | Group 4 |
|---|---|---|---|---|
| | (n = 5) | (n = 5) | (n = 5) | (n = 5) |
| Period 1 | C | Lutalin | Lutrell | Lutalin + |
| | | | | Silafett |
| Period 2 | Lutalin | C | Lutalin + | Lutrell |
| | | | Silafett | |
| Period 3 | Lutalin + | Lutrell | C | Lutalin |
| | Silafett | | | |
| Period 4 | Lutrell | Lutalin + | Lutalin | C |
| | | Silafett | | |

Assignation to the table of results (section f) hereinbelow: C = Control = T1, Lutrell = T2, Lutalin = T3 and Lutalin plus Silafett = T4.

Each period is 21 days; i.e. an adaptation time of 14 days is followed by a measuring period of 7 days. All four test rations are based on the same basic components.

### c) Animals

Each treatment is tested according to the above experimental design on 20 cows. A further 4 cows are assigned to the test groups by way of reserve animals. At the beginning of the experiment, the test cows are in the middle lactation stage. The mean milk yield at the beginning of the experiment is approx. 32 kg FCM per day. The test cows (German Holstein) are selected from the experimental station's herd (herd performance approx. 11 000 kg milk, 3.9% fat, 3.4% protein) and assigned to the four groups by the criteria
lactation number (1 and above)
milk fat content (based on the two preceding milk yield recordings)
day of lactation and milk yield
   with the aim of adjusting comparable group averages.

### d) Feeding

All cows are provided with the same total mixed ration (TMR), which is fed once per day ad libitum. The TMR is composed of corn silage, grass silage, hay and approx. 45% concentrate. The TMR target values are shown in the table which follows. This TMR ensures that the cows are provided with sufficient nutrients for a milk yield of 33 kg if they take in approx. 21 kg DM of this feed.

| | uXP | XP | XL | XF | NEL |
|---|---|---|---|---|---|
| | g/kg T | g/kg T | g/kg T | g/kg T | MJ/kg T |
| Total | 155 | 155 | 30 | 180 | 6.9 |

Crude nutrient content of the TMR as per Weender analysis (uXP = utilizable crude protein, XP = crude protein, XL = crude fat, XF = crude fiber and NEL = net energy lactation).

The three CLA formulations are first used for preparing pelleted premixes with a milk production feed. Suitable amounts of these premixes (corresponding to 1 kg per animal per day) are then admixed by hand to the TMR in the feed troughs with the aim of obtaining an amount of in each case 16.7 g of the pure substances in 20 kg DM of the TMR. The control group receives the same concentrate in the same amount, without CLA.

### e) Sampling and Measured Parameters

### Feed

For determining the dry matter, one daily TMR sample is taken, and the samples are combined to give a cumulative sample per measuring period (7 days). This cumulative sample is used for determining the crude nutrient contents as per Weender analysis and the energy content as per the Hohenheimer feed value test.

### Cows

The TMR uptake, the milk yield and the live weight are recorded daily (individually) for each cow. During the 7-day measuring period, milk samples are taken three times in the form of aliquots from the evening and the morning milk and tested for the fat, total protein (Nx6.38), lactose and urea contents and the somatic cell count by the Baden-Württemberg milk testing organization. An additional milk test is performed in the middle of the first and the second week of each experimental period (preparation phase). A cumulative milk sample which comprises the 7-day measuring period is additionally formed for each cow and each treatment and then frozen so as to be able to optionally perform a fatty acid analysis of the milk fat therein.

### f) Experimental Results

TABLE
Effect of Lutalin and Lutrell in pelleted feed on milk fat depression
in dairy cows fed with 5 g of trans-10/cis-12 CLA

| | | Milk | Fat | Protein | Protein | Fat | |
|---|---|---|---|---|---|---|---|
| Treatment | | kg | kg | kg | % | % | MFD |
| T1 = Control | Mean | 34.4 | 1.310 | 1.153 | 3.40 | 3.86 | 0% |
| | SD | 6.56 | 0.232 | 0.170 | 0.38 | 0.58 | |
| T2 = Lutrell | Mean | 34.6 | 1.190 | 1.146 | 3.36 | 3.52 | −9% |
| | SD | 7.60 | 0.220 | 0.200 | 0.38 | 0.59 | |
| T3 = Lutalin | Mean | 35.1 | 1.218 | 1.156 | 3.34 | 3.52 | −9% |
| | SD | 7.27 | 0.218 | 0.183 | 0.37 | 0.54 | |
| T4 = Lutalin | Mean | 34.7 | 1.205 | 1.151 | 3.36 | 3.50 | −9% |
| plus/Silafett | SD | 6.56 | 0.224 | 0.167 | 0.37 | 0.46 | |

SD: Standard deviation

Surprisingly, treatments T3 and T4 resulted in the same milk fat depression as treatment T2, although the active substance CLA in T3 and T4 had been admixed in a non-rumen-protected form into the milk production ration. Accordingly, pelleting protected the active substance CLA of T3 and T4 in the rumen to the same extent as this is known from Lutrell, and has also been demonstrated by T2.

### Example 2: Study into the Rumen Stability of Various Pelleted CLA Feed Formulations

Stability in the rumen is the essential prerequisite for the milk fat depression by CLA-containing feeds. To this end, the feeds tested in example 1 (75% corn plus 25% extracted soybean meal) were mixed with 1.66% Lutalin or 5% Lutrell, and the mixture was pelleted or extruded. The pellets and extrudates produced were then incubated in vitro for 4, 12 and 24 hours. After the predetermined times, the incubation was stopped, and all of the contents were transferred into a vessel and freeze-dried. The CLA content was subsequently determined on this freeze-dried sample material.

### a) Pelleting and Extrusion

Preparation of the meal: Corn and extracted soybean meal were weighed in together, ground and mixed in a horizontal mixer. Lutalin or Lutrell were added and mixed in. The mixing time amounted to 15 minutes. These two mixtures were divided in each case. 80 kg were pelleted and 30 kg were extruded.

Pelleting press: Manufacturer Simon Heesen, horizontal die, 3.3×35 mm, nominal capacity 300 kg/hr.
Extruder: Werner & Pfleiderer 37, die 2×0 3.9 mm, nominal capacity up to 50 kg/hr.
Pelleting: Pelleting temperatures of 67-68.5° C. were reached.
Extrusion: An extrusion temperature of 85° C. was reached, approximately 14% of water having been added. A residual moisture of 10 to 12% has been found in the end product.

### b) CLA Formulations Employed

(1) Lutalin®: (CLA-containing commercial product. Composition see example 1.
(2) Lutrell® Pure: CLA-containing commercial product. Composition see example 1

### c) Experimental Set-Up

4 test variants (see table hereinbelow) were tested.

Variant 1 (T1) One pellet and contains the CLA formulation “Lutalin”
Variant 2 (T2) One pellet and contains the CLA formulation “Lutrell”
Variant 3 (T3) One extrudate and contains the CLA formulation “Lutalin”
Variant 4 (T4) One extrudate and contains the CLA formulation “Lutrell”

| | T1 | T2 | T3 | T4 |
|---|---|---|---|---|
| Soya/corn mixture | 983.3* | 950 | 983.3 | 950.0 |
| Lutrell | | 50 | 0.0 | 50 |
| Lutalin | 16.7 | | 16.7 | 0 |
| Total | 1000 | 1000 | 1000 | 1000 |

*Data in grams
Feed: 1 kg supplement composed of 25% extracted soybean meal and 75% corn meal.

An analysis performed immediately after the processing step revealed that the active substance CLA survived the pelleting process very well, but that extrusion resulted in an activity loss of approx. 40%.

### d) Incubation: Determination of In-Vitro Degradation Products in the Hohenheimer Feed Value Test (In-Vitro Test)

The Hohenheimer feed value test (HFT) is an in-vitro method for estimating the energetic feed value of ruminant feed. An adapted variant makes it possible also to test active substances in the HFT in respect of their rumen stability, when incubation is stopped after defined periods of time and the remainder of the active substance in the incubation solution is analyzed.

The Hohenheimer feed value test (HFT) is carried out as per the prescribed VDLUFA method (Methodenbuch [Methods book] vol. III, chapter 25.1).

The incubation period depends on the purpose of the test in question; for the degradation of CLA formulations, it is expediently between 4 and 24 hours. The in-vitro system is stable for up to 48 hours; beyond this point in time, however, deviations from a physiological course of the fermentation must be expected. In each case 500 mg of the test variants T1 to T4 (corresponding to approx. 5 mg CLA, or approx. 3 mg for the extruded variants) were weighed in. The incubation period amounted to 4, 12 and 24 h. A triple determination (3 incubation vessels per test variant T1 to T4) was carried out for each incubation period.

After the relevant times, the incubation was stopped by cooling in ice-water. Each of the rumen fluid/buffer mixtures was quantitatively transferred into glass vessels, which are suitable for the subsequent freeze-drying step. The drying vessels are weighed empty, so that, after drying, it was possible to determine the residual weight and use the data for a subsequent quantitative analysis. The freeze-drying system used was a Gamma 1-20 from Christ, 37507 Osterode.

### e) Analytical Method: Modification of AM/00887/01

Owing to the specific matrix and the low CLA content, the processing as described in the analytical method (see Chapter 7.4.2) had to be modified as described hereinbelow. The modifications performed are shown in italics.

The sample is weighed or introduced into a 250-ml Erlenmeyer flask as described in 7.4.2; thereafter, the stated amounts of BHT, sodium ascorbate and water are added.

Then, a spatula-tip full of the enzyme Pronase is added, and the Erlenmeyer flask is placed for 15 minutes into an ultrasonic bath at a temperature of 60° C.

After adding the amounts of ethanol and acetic acid which are described in 7.4.2, the sample is returned for 15 minutes into the ultrasonic bath at a temperature of 60° C.

After cooling to room temperature, extraction solution cyclohexane/ethyl acetate 80:20 (v/v) is added. However, only 50 ml of the extraction solution are used, instead of normally 100 ml.

Thereafter, the sample is stirred for 30 minutes on the magnetic stirrer as described in 7.4.2, 70 ml saturated sodium chloride solution are added, and stirring is continued for 10 minutes. Thereafter, the stirrer is switched off, and the mixtures are left to stand until the phases separate.

3 ml of the organic phase are withdrawn, the solvents cyclohexane and ethyl acetate are removed in the stream of nitrogen, and the residue is taken up in 2 ml of the extraction solution cyclohexane/ethyl acetate 80:20 (v/v) (concentration).

An aliquot of the resulting sample solution is directly filtered, by a disposable 0.45-μm-filter, into an HPLC vial.

The injection volume is 20 μl, instead of normally 10 μl.

### f) Experimental Results

### Retention (%) CLA-Total (as the Methyl Ester and as Free Fatty Acid)

| | Amount | mg retention | | | Retention in % of starting weight | | | |
|---|---|---|---|---|---|---|---|---|
| | weiged in, mg | Retention time, h | | | Retention time, h | | | |
| | 0 | 4 | 12 | 24 | 0 | 4 | 12 | 24 |
| Pellet Lutalin | 5.1 | 1.49 | 0.32 | 0.09 | 100% | 29% | 6% | 2% |
| Pellet Lutre II | 5.3 | 2.09 | 0.71 | 0.14 | 100% | 39% | 13% | 3% |
| Extrudate Lutalin | 3.05 | 2.76 | 1.22 | 0.16 | 100% | 91% | 40% | 5% |
| Extrudate Lutrell | 2.95 | 1.10 | .54 | 0.54 | 100% | 37% | 18% | 18% |

In this in-vitro experiment, it was possible to reproduce the identical milk fat depression of pelleted Lutalin versus pelleted Lutrell, which had already been found, surprisingly, in example 1, as the CLA retentions measured in the two pelleted variants T1 and T2 were identical. Therefore, using this test, it is possible to assess with certainty at least those variants which are based on the same feed and which merely differ in a physical treatment. Surprisingly, the extruded variants T3 and T4 showed in each case substantially higher retention rates—after 4 and 12 h, and after 12 and 24 h, respectively—than the pelleted variants (cf. FIG. 1).

It can therefore be concluded that Lutalin (non-rumen-stabilized CLA) in extruded form, too, has an at least equivalent rumen stability as a comparable Lutrell extrudate (CLA which has been rumen-stabilized by stabilizing additives).

Reference is expressly made to the disclosure of the publications mentioned herein.

1-15. (canceled)

16. A method comprising administering to a lactating ruminant an uncoated pelleted ruminant feed, comprising, in pelleted form, a mixture of at least one solid particulate feed component with at least one rumen-labile constituent which has been added to the mixture, wherein the at least one rumen-labile constituent is at least one conjugated linolenic acid (CLA) or a salt thereof selected from the group consisting of:
a) cis/trans-9,11-linoleic acid;
b) cis/trans-8,10-linoleic acid;
c) cis/trans-11,13-linoleic acid;
d) cis/trans-10,12-linoleic acid; and
e) mixtures of at least two of a), b), c) and d).

17. The method of claim 16, wherein the CLA is selected from the group consisting of:
a) 9-cis,11-trans-linoleic acid;
b) 10-trans,12-cis-linoleic acid; and
c) mixtures of a) and b).

18. The method of claim 16, wherein the CLA is present as a pure substance or as a natural or synthetic mixture of substances, comprising at least one of these CLAs.

19. The method of claim 16, wherein the amount of added rumen-labile constituent is in the range of from 0.1 to 20% by weight, based on the total weight of the mixture to be pelleted.

20. The method of claim 16, wherein the uncoated pelleted ruminant feed is prepared by a process comprising:
a) mixing at least one solid particulate feed component with the at least one rumen-labile constituent;
b) optionally conditioning the resulting mixture;
c) compressing the optionally conditioned mixture in a pelleting, extruding or expanding device with the action of pressure and optionally steam to give pellets; and
d) optionally cooling and/or drying the resulting pellets and optionally crumbling the pellets.

21. A method of altering the milk fat concentration in milk which is produced by a lactating ruminant, comprising administering to a lactating ruminant an uncoated pelleted ruminant feed, comprising, in pelleted form, a mixture of at least one solid particulate feed component with at least one rumen-labile constituent which has been added to the mixture, wherein the at least one rumen-labile constituent is at least one conjugated linolenic acid (CLA) or a salt thereof selected from the group consisting of:
a) cis/trans-9,11-linoleic acid;
b) cis/trans-8,10-linoleic acid;
c) cis/trans-11,13-linoleic acid;
d) cis/trans-10,12-linoleic acid; and
mixtures of at least two of a), b), c) and d).

22. The method of claim 21, wherein the lactating ruminant is a cow, goat or sheep.

23. The method of claim 16, wherein the lactating ruminant is a cow, goat or sheep.

24. The method according to claim 16, wherein the amount of added rumen-labile constituent is in the range of from 1 to 15% by weight, based on the total weight of the mixture to be pelleted.

25. The method according to claim 16, wherein the amount of added rumen-labile constituent is in the range of from 2 to 10% by weight, based on the total weight of the mixture to be pelleted.

26. The method according to claim 16, wherein the amount of added rumen-labile constituent is in the range of from 3 to 5% by weight, based on the total weight of the mixture to be pelleted.

27. The method according to claim 16, wherein the rumen-labile constituent is administered to the lactating ruminant in an amount sufficient to effect milk fat depletion (MFD).
